(19) United States
(12) Patent Application Publication
(10) Pub. No.: US 2008/0177267 A1
(43) Pub. Date: Jul. 24, 2008

(54) RETROFIT CLAMP WITH RFID TAG FOR A SURGICAL TOOL

(76) Inventors: Daniel L. Sands, Warsaw, IN (US); Michael D. Westrick, Fort Wayne, IN (US)

Correspondence Address: TAYLOR & AUST, P.C., P.O. Box 560, 142. S Main Street, Avilla, IN, 46710, US

(21) Appl. No.: 12/016,516

(22) Filed: Jan. 18, 2008

Related U.S. Application Data
(60) Provisional application No. 60/885,759, filed on Jan. 19, 2007.

(51) Int. Cl. A61B 17/16; G06K 19/06; F16M 13/00

(52) U.S. Cl. 606/80; 235/492; 248/542

(57) ABSTRACT

A surgical tool assembly includes a surgical tool having an outer surface, and a clamp having an opening which is sized and shaped to generally match with and lie closely around a selected portion of the outer surface of the surgical tool. The clamp has an RFID tag therein.

### CROSS REFERENCE TO RELATED APPLICATIONS

This is a non-provisional application based upon U.S. provisional patent application Ser. No. 60/885,759, entitled “RETROFIT CLAMP WITH RFID TAG FOR A SURGICAL TOOL”, filed Jan. 19, 2007, which is incorporated herein by reference.

### BACKGROUND OF THE INVENTION

1. Field of the Invention

The present invention relates to surgical instruments, and, more particularly, to radio frequency identification (RFID) tags used with such surgical instruments.

2. Description of the Related Art

RFID tags are well known throughout industry, and are being increasingly utilized for supply chain management, inventory management, and logistic control. These tags can be written to and read from a handheld transceiver or fixed portal. Small glass encapsulated low frequency tags are currently being utilized in surgical tools and implantable devices.

Surgical tools such as orthopaedic instruments may be placed within cases for storage and transport. It may be desirable to track data associated with a particular instrument, the location of particular instruments, and inventory sets of instruments within a case. This can be done manually, but of course is tedious, time consuming and expensive.

Surgical tools may be configured as original equipment to be automatically tracked or inventoried using RFID tags. For example, it could be possible to embed an RFID tag within a non-load bearing portion of a surgical tool from an original equipment manufacturer (OEM), such as a handle, etc.; however, there is simply no thought or provisions for using RFID tags in a retrofit application for such surgical tools.

What is needed in the art is a way of retrofitting existing surgical tools with RFID tags.

### SUMMARY OF THE INVENTION

The present invention provides a retrofit clamp which has an internal RFID tag and clamps around a selected portion of a surgical tool.

The invention in one form is directed to a surgical tool assembly, including a surgical tool having an outer surface, and a clamp having an opening which is sized and shaped to generally match with and lie closely around a selected portion of the outer surface of the surgical tool. The clamp has an RFID tag therein.

The invention in another form is directed to a retrofit clamp for use with a surgical tool, including a housing having an opening which is sized and shaped to generally match with and lie closely around a selected portion of an outer surface of the surgical tool. The clamp has an RFID tag therein.

An advantage of the present invention is that a surgical tool can be retrofitted with an RFID tag for inventory, tracking and logistics purposes.

Another advantage is that the clamp can be easily modified to fit various sized and shaped surgical tools.

Yet another advantage is that the clamp can be attached to a surgical tool quickly and easily, with or without the use of tools (depending on the configuration).

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of one embodiment of the invention with the clamp in an open position;

FIG. 2 is another perspective view of the clamp shown in FIG. 1; and

FIG. 3 is a perspective view of another embodiment of the invention with the clamp in a closed position.

Corresponding reference characters indicate corresponding parts throughout the several views. The exemplifications set out herein illustrate embodiments of the invention, and such exemplifications are not to be construed as limiting the scope of the invention in any manner.

### DETAILED DESCRIPTION OF THE INVENTION

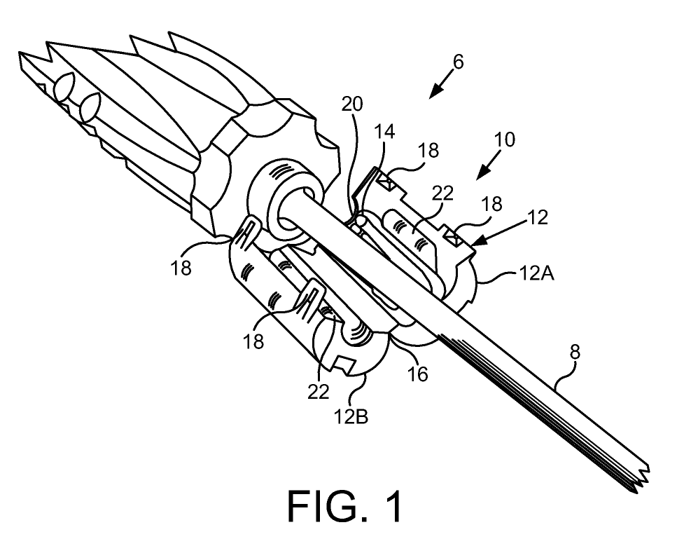

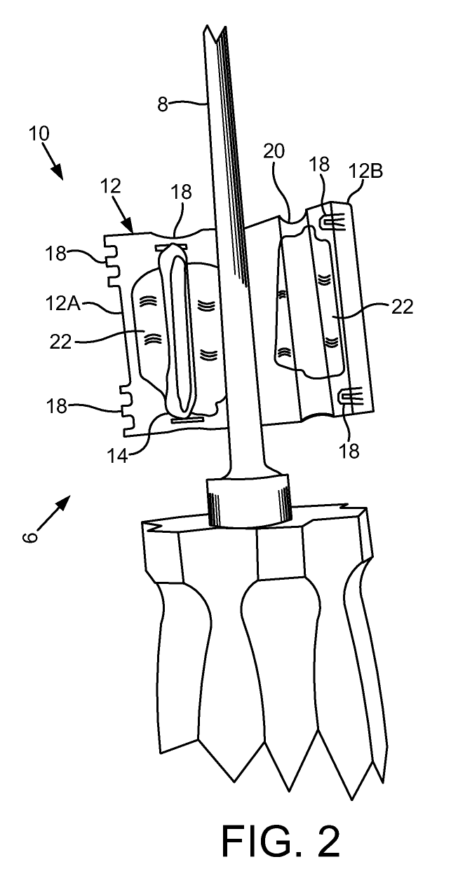

Referring now to the drawings, and more particularly to FIGS. 1 and 2, there is shown an embodiment of a surgical tool assembly 6 of the present invention, including a surgical tool 8 and retrofit clamp 10. Surgical tool 8 is shown in the form of a surgical screwdriver, but could be configured as a different type of surgical tool, such as a hammer, wrench, milling cutter, reamer, rasp, drill, alignment device, orientation device, etc.

Retrofit clamp 10 generally includes a housing 12 and RFID tag 14. Housing 12 is a multiple part housing, with a first housing part 12A and second housing part 12B which are hinged together with a living hinge 16 along one edge and snap fit together with a snap fit arrangement 18 along an opposite edge. Housing parts 12A and 12B in general form a clam shell arrangement (not numbered) which is clamped around a selected portion of surgical tool 8. To that end, housing 12 includes an opening 20 which is sized and shaped to match with and lie closely adjacent to a selected portion of surgical tool 8. The particular overall size and shape of housing 12 and opening 20 varies depending upon the particular type of tool with which clamp 10 is to be retrofitted.

RFID tag 14 can be a conventional tag or a specially designed tag, again depending upon the application. RFID tag 14 may be held within housing 12 using suitable techniques, such as using potting material 22, etc. RFID tag 14 can be placed at any desired or suitable location within housing 12. In one embodiment, RFID tag 14 is placed within housing 12 to lie adjacent to and electromagnetically couple with surgical tool 8, thereby utilizing surgical tool 8 as an antenna for RFID tag 14. RFID tag 14 can be biased against surgical tool 8 using a resilient potting material, spring arrangement, or the like.

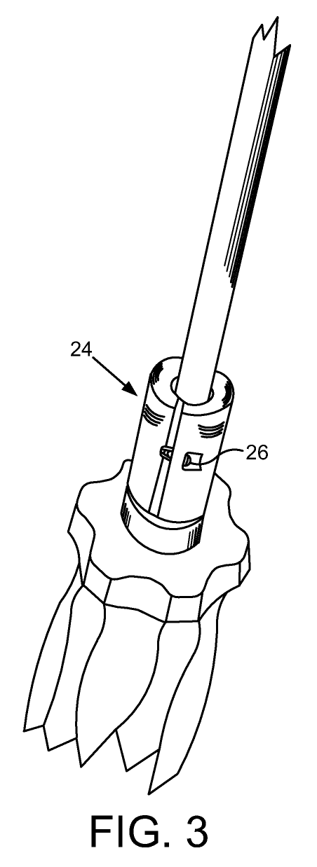

FIG. 3 shows another embodiment of a retrofit clamp 24 of the present invention which is sized and shaped similar to clamp 10, but includes a two-part housing which is screwed together along opposite side edges using fasteners 26, rather than having a hinge and snap fit arrangement.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A surgical tool assembly, comprising:
a surgical tool having an outer surface; and
a clamp having an opening which is sized and shaped to generally match with and lie closely adjacent to a selected portion of said outer surface of said surgical tool, said clamp having a radio frequency identification (RFID) tag therein.

2. The surgical tool assembly of claim 1, wherein said clamp is clamped around said outer surface of said surgical tool.

3. The surgical tool assembly of claim 1, wherein said RFID tag lies adjacent to said opening.

4. The surgical tool assembly of claim 3, wherein said RFID tag electromagnetically couples with said tool.

5. The surgical tool assembly of claim 1, wherein said clamp has a housing defining said opening.

6. The surgical tool assembly of claim 5, wherein said housing is a multiple part housing.

7. The surgical tool assembly of claim 6, wherein said multiple parts of said housing are coupled together using a snap fit arrangement.

8. The surgical tool assembly of claim 6, wherein said multiple parts of said housing are coupled together using fasteners.

9. The surgical tool assembly of claim 6, wherein said housing has a clam shell arrangement.

10. The surgical tool assembly of claim 9, wherein said housing has a living hinge.

11. The surgical tool assembly of claim 1, wherein said surgical tool comprises one of a surgical screwdriver, hammer, wrench, milling cutter, reamer, rasp, drill, alignment device, and orientation device.

12. A retrofit clamp for use with a surgical tool, comprising:
a housing having an opening which is sized and shaped to generally match with and lie closely adjacent to a selected portion of an outer surface of the surgical tool, said clamp having a radio frequency identification (RFID) tag therein.

13. The clamp of claim 12, wherein said RFID tag lies adjacent to said opening.

14. The clamp of claim 12, wherein said RFID tag is configured to electromagnetically couple with the tool.

15. The clamp of claim 12, wherein said housing is a multiple part housing.

16. The clamp of claim 15, wherein said multiple parts of said housing are coupled together using a snap fit arrangement.

17. The clamp of claim 15, wherein said multiple parts of said housing are coupled together using fasteners.

18. The clamp of claim 15, wherein said housing has a clam shell arrangement.

19. The clamp of claim 18, wherein said housing has a living hinge.
